(19) United States
(12) Patent Application Publication
(10) Pub. No.: US 2020/0281773 A1
(43) Pub. Date: Sep. 10, 2020

(54) EARPLUG WITH ENHANCED WEARING COMFORTABILITY

(71) Applicant: CONCRAFT HOLDING CO., LTD., Grand Cayman (KY)

(72) Inventors: Jie-Ru CHEN, New Taipei City (TW); Tzu-Ching TSAI, New Taipei City (TW)

(21) Appl. No.: 16/429,803

(22) Filed: Jun. 3, 2019

(30) Foreign Application Priority Data
Mar. 5, 2019 (TW) 108107177

(51) Int. Cl. A61F 11/08

(52) U.S. Cl. CPC A61F 11/08; A61F 2011/085

(57) ABSTRACT

An earplug, which is inserted and worn inside a person's ear canal, consists of a main guide body provided with a compression portion, a supporting stem provided with an air passage and used to support the main guide body, and a filler material provided with plasticity that fills the compression portion. A filling cavity is provided inside the compression portion that interconnects with the air passage and enables filling with the filler material, and causes an air chamber that connects to the air passage to form inside the compression portion, enabling the compression portion to form an original state. The compression portion is in a compressed state when pressed and constricted, and air circulating in the air passage is used to cause changes in the compression portion between its original state and compressed state, which enables enhancing comfortability when wearing the earplug.

### BACKGROUND OF THE INVENTION

### (a) Field of the Invention

The present invention relates to an earplug, and more particularly to an earplug that is inserted and worn inside a person's ear canal, and uses circulation of air to enhance comfortability when wearing.

### (b) Description of the Prior Art

Earplugs are mainly used for placing inside a person's ear canal to completely cut off noise from entering the ear to achieve protection of the person's ear canal.

Commonly seen earplugs products mainly use an integral body formed from foam or silicone to produce a multi-disc umbrella shaped body that facilitates placement inside a person's ear canal to achieve a protection effect. However, after insertion into the ear canal for a long period of time, these types of earplug products easily result in discomfort in the person's ear canal, and is unable to satisfy various different types of ear canals.

Hence, presently, various different inflatable earplug products have appeared on the market, such as U.S. Patent No. 20090173353 or, U.S. Patent No. 2015282990, These types of inflatable ear protector products directly fill bags with air, however, after inflating, these types of inflatable ear protector products do not allow separate adjustment of the inflation state of the bags, which easily causes discomfort when wearing the ear protector.

U.S. Pat. No. 9,326,059 discloses another type of inflatable ear protector product, which is configured with a plurality of bags that are assembled and mutually connected using an encircling center method. When inflating the ear protector, the plurality of bags are concurrently inflated, however, during inflation, this type of inflatable ear protector product similarly disallows the separate adjustment of the inflation state of the plurality of bags, which not only easily causes discomfort when wearing the ear protector, but is also unsuitable for various different types of ear canals, and thus unable to satisfy various different ear canals, resulting in poor application effectiveness.

Accordingly, the technical problems the present invention intends to resolve and objectives thereof involves providing an earplug with enhanced wearing comfortability.

### SUMMARY OF THE INVENTION

The main object of the present invention lies in providing an earplug, and more particularly to an earplug that is inserted and worn inside a person's ear canal, and uses circulation of air to enhance comfortability when wearing.

In order to achieve the aforementioned object, an earplug with enhanced wearing comfortability of the present invention comprises a main guide body, a supporting stem, and a filler material provided with plasticity. The main guide body further comprises a guide portion, a compression portion that extends from the guide portion, and a stop portion that extends from the compression portion. A connecting cavity is provided inside the guide portion, a filling cavity is provided inside the compression portion, and an opening portion is provided inside the stop portion. The filling cavity interconnects the connecting cavity and the opening portion. The supporting stem is provided with a connecting section that enables embedding into the connecting cavity, an extended section that extends from the connecting section and is positioned inside the filling cavity, and a securing section that extends from the extended section and embeds within the opening portion. Moreover, the securing section is provided with an air passage, which enables air to circulate between the exterior of the main guide body and the interior of the filling cavity by passing through the air passage. The filler material fills the interior of the filling cavity to enable an air chamber that can contain air to form therein, thereby enabling air to enter the air chamber through the air passage for the compression portion to maintain its original state. Accordingly, when the compression portion is pressed and constricted, the compression portion compresses the air within the air chamber through the filler material, thereby expelling the air through the air passage and causing the compression portion to form a compressed state. After releasing the pressure on the compression portion, air enters the air chamber through the air passage and squeezes the filler material, which causes the compression portion to return to its original state from a compressed state.

In a second embodiment, the compression portion is further assembled from a first compression area connected to the guide portion and a second compression area that extends from the first compression area and is connected to the stop portion.

In the second embodiment, the filling cavity is further is assembled from a first filling cavity that is positioned inside the first compression area and a second filling cavity that is positioned inside the second compression area and connects with the air passage; moreover, the first filling cavity and the second filling cavity are interconnected.

In the second embodiment, the filler material fills the first filling cavity and a portion of the second filling cavity, and the air chamber is formed inside the second filling cavity.

In the embodiments, a mutually embedding structure is formed between the opening portion and the securing section.

In the embodiments, the embedding structure comprises a first embedding portion positioned at the opening portion and a second embedding portion positioned on the securing section and embedded into the first embedding portion.

In the embodiments, both the guide portion and the stop portion assume an umbrella form.

In the embodiments, the filler material is formed from silicone material.

In the embodiments, the supporting stem further comprises a holding section extending from the securing section to the outer side of the opening portion, and the holding section extends to the exterior of the main guide body.

The earplug of the present invention has the following advantages compared to the prior art:

When inserting the earplug into a person's ear canal, the compression portion compresses in accordance with changes in the shape of the person's ear canal, and the shape of the compression portion changes through air adjustment to achieve enhanced comfortability when wearing the earplug.

To enable a further understanding of said objectives and the technological methods of the invention herein, a brief description of the drawings is provided below followed by a detailed description of the preferred embodiments.

### BRIEF DESCRIPTION OF THE DRAWINGS

FIG. 1 is a cross-sectional schematic view of a first embodiment aspect of the present invention.

FIG. 2 is a three-dimensional view of a second embodiment aspect of the present invention.

FIG. 3 is planar schematic view of the second embodiment aspect of the present invention.

FIG. 4 is a cross-sectional schematic view of a first viewing angle of the second embodiment of the present invention.

FIG. 5 is a cross-sectional schematic view of a second viewing angle of the second embodiment aspect of the present invention.

FIG. 6 is a cross-sectional schematic view of a decomposed state of the first viewing angle of the second embodiment aspect of the present invention.

FIG. 7 is schematic view of the second embodiment of the present invention placed inside a person's ear canal.

### DETAILED DESCRIPTION OF THE PREFERRED EMBODIMENTS

Regarding the detailed content and structural means of present invention, please refer to the description of the diagrams as follows:

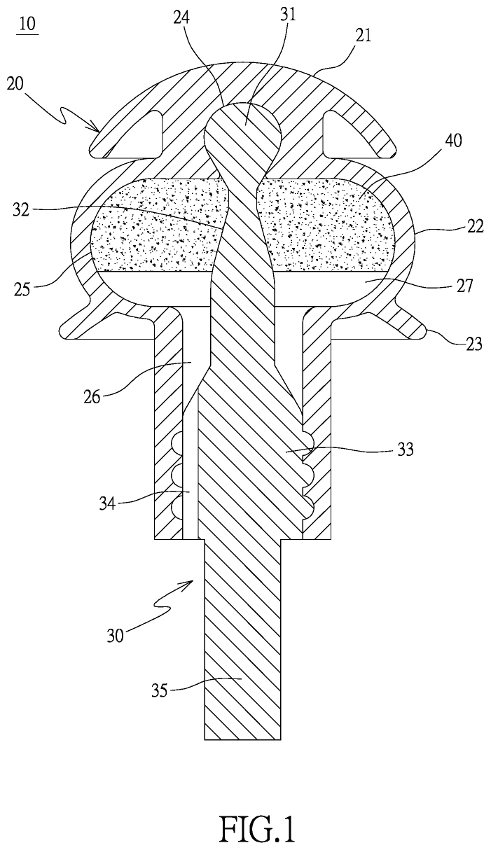

Referring to FIG. 1, which shows an earplug with enhanced wearing comfortability of the present invention, wherein an earplug 10 comprises a main guide body 20, a supporting stem 30, and a filler material 40.

The main guide body 20 further comprises a guide portion 21, a compression portion 22 that extends from the guide portion 21, and a stop portion 23 that extends from the compression portion 22, all arranged in an axial configuration. The exterior of the guide portion 21 assumes an umbrella form, and a connecting cavity 24 is provided inside the guide portion 21. A filling cavity 25 is provided inside the compression portion 22, the exterior of the stop portion 23 assumes an umbrella form, and an opening portion 26 is provided inside the stop portion 23. The filling cavity 25 interconnects the connecting cavity 24 and the opening portion 26.

The supporting stem 30 that extends along the same axial direction as the main guide body 20 is provided with a connecting section 31 that enables embedding into the connecting cavity 24, an extended section 32 that extends from the connecting section 31 and is positioned inside the filling cavity 25, and a securing section 33 that extends from the extended section 32 and embeds within the opening portion 26. Moreover, the securing section 33 is provided with an air passage 34, which enables air to pass therethrough (not shown in the drawing) and circulate between the exterior of the main guide body 20 and the interior of the filling cavity 25. In addition, a holding section 35 further extends outward to the exterior of the main guide body 20 from the securing section 33.

The filler material 40 is provided with plasticity and fills the interior of the filling cavity 25 to enable an air chamber 27 to form therein and contain air. Air can enter the air chamber 27 through the air passage 34 for the compression portion 22 to maintain an original state.

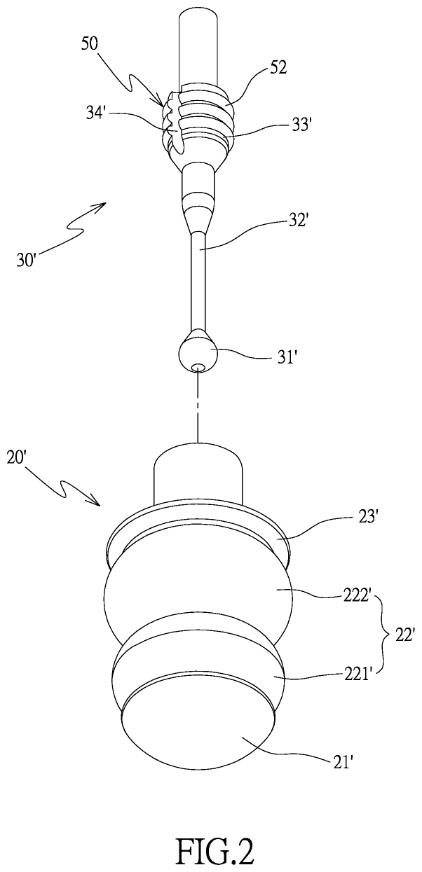

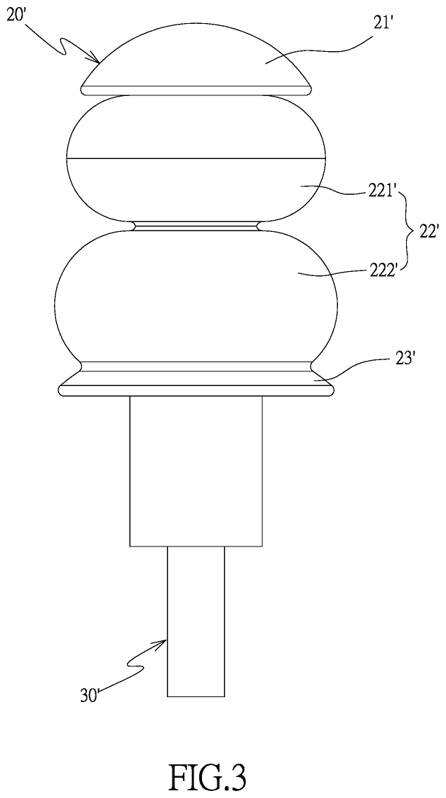

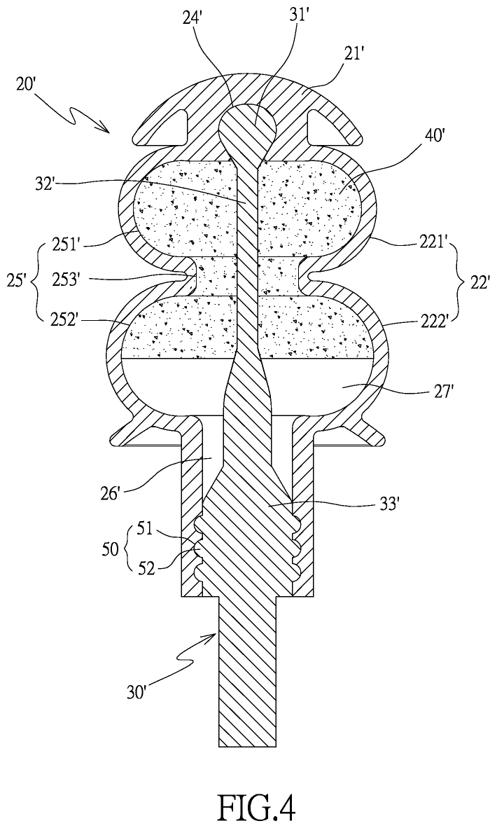

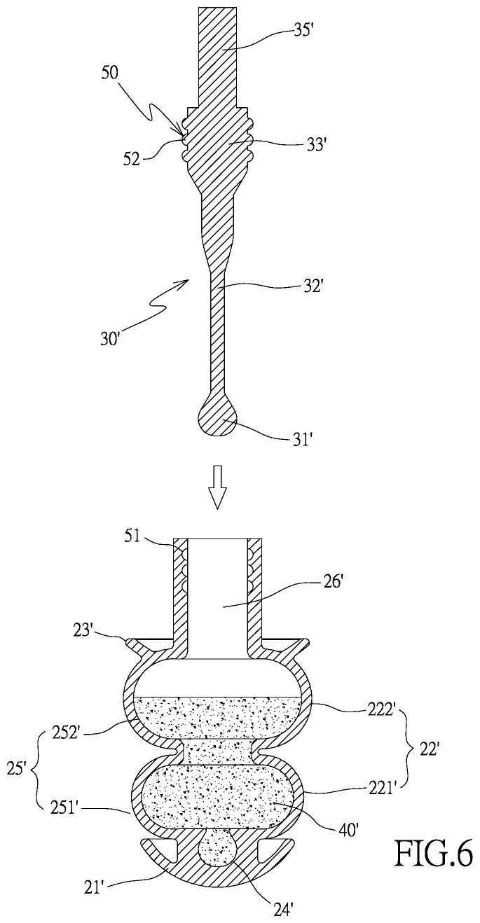

Referring further to FIGS. 2 to 6, which show a second embodiment aspect of the earplug 10, and in this embodiment a compression portion 22′ further comprises a first compression area 221′ that extends from a guide portion 21′ and a second compression area 222′ that extends from the first compression area 221′.

The second compression area 222′ is connected to a stop portion 23′, and a filling cavity 25′ further comprises a first filling cavity 251′ that is positioned inside the first compression area 221′ and a second filling cavity 252′ that is positioned inside the second compression area 222′, wherein a passage cavity 253′ provides interconnection between the first filling cavity 251′ and the second filling cavity 252′. The diameter of the passage cavity 253′ is smaller than the diameters of both the first filling cavity 251′ and the second filling cavity 252′, and the diameter of the first filling cavity 251′ is slightly smaller than that of the second filling cavity 252′. The second filling cavity 252′ is connected to an opening portion 26′, and a filler material 40′ respectively fills the first filling cavity 251′, the passage cavity 253′, and the interior of the second filling cavity 252′. Moreover, an air chamber 27′ is positioned inside the second filling cavity 252′ and connects to an air passage 34′.

It is worth mentioning that the filler material 40′ in the second embodiment is formed from silicone material, and the silicone filler material 40′ is first heated to form a liquid, after which the liquid silicone material is injected into the interior of the main guide body 20′. It is important to explain that the liquid silicone material does not completely fill the interior of the second filling cavity 252′ when injecting the liquid silicone material into the interior of the main guide body 20′. After injecting the liquid silicone material, the supporting stem 30′ is disposed into the interior of the main guide body 20′ before the liquid silicone material has cooled down to enable inserting and embedding a connecting section 31′ of the supporting stem 30′ into a connecting cavity 24′ while enabling an extended section 32′ to extend into the filling cavity 25′.

In addition, an embedding structure 50 is further configured between a securing section 33′ and the opening portion 26′, wherein the embedding structure 50 comprises: a first embedding portion 51 positioned at the opening portion 26′ and a second embedding portion 52 positioned on the securing section 33′ and embedded into the first embedding portion 51. Accordingly, when the supporting stem 30′ is disposed into the interior of the main guide body 20′, apart from embedding the connecting section 31′ into the connecting cavity 24′, the supporting stem 30′ is also embedded into the opening portion 26′ by means of the securing section 33′, thereby enabling the supporting stem 30′ to effectively embed into the main guide body 20′ and preventing separation from occurring between the supporting stem 30′ and the main guide body 20′.

After disposing the supporting stem 30′ into the interior of the main guide body 20′, a silicone material is caused to solidify inside the filling cavity 25′ and form the filler material 40′ using a cooling or photocuring method, which also causes the air chamber 27′ to form within the second filling cavity 252′, thereby enabling air to pass through the air passage 34′ on the supporting stem 30′ from the exterior of the main guide body 20′ and enter the air chamber 27′, which further enables the compression portion 22′ to maintain its original state.

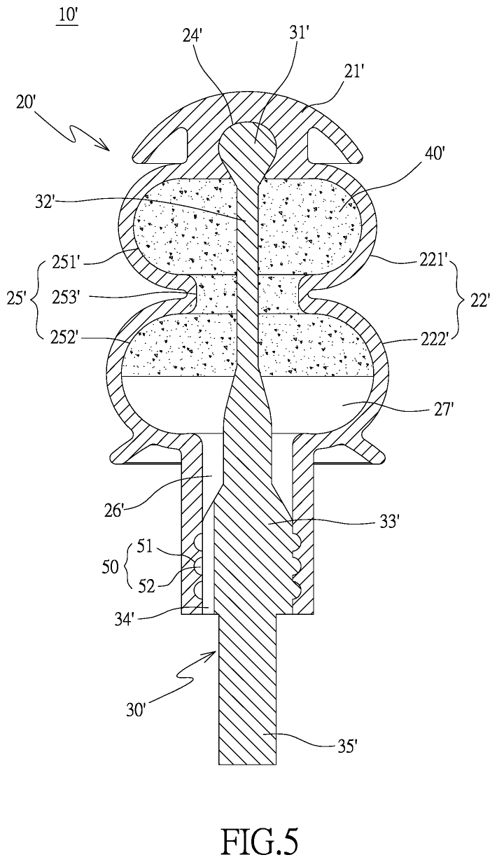

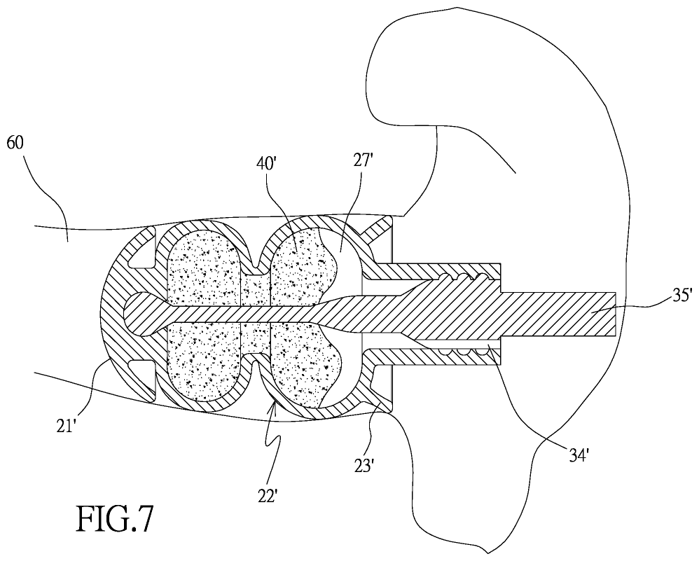

Referring to FIGS. 5 to 7, when an earplug 10′ is inserted inside a person's ear canal 60, it is of prime importance to first use the guide portion 21′ to enter the person's ear canal 60, which is followed by the compression portion 22′ gradually coming into contact with the interior of the person's ear canal 60, whereupon the ear wall presses against the compression portion 22′ causing deformation thereof and compression of the filler material 40′ within the filling cavity 25′. And because the filler material 40′ is provided with plasticity, thus, the filler material 40′ also deforms within the filling cavity 25′. After deforming, the filler material 40′ produces a pushing force toward the air chamber 27′ that squeezes and expels the air therein to the exterior of the main guide body 20′ through the air passage 34′, thereby causing the compression portion 22′ to form a compressed state and enabling the stop portion 23′ to block the entrance area of the person's ear canal 60. Accordingly, apart from enabling the compression portion 22′ to produce a more favorable contact effect with the person's ear canal 60, the earplug 10′ achieves better airtightness within the person's ear canal 60. In addition, compression of the compression portion 22′ by the person's ear canal 60 compresses the filler material 40′, which then squeezes and expels the air out to effectively reduce the pressure within the earplug 10′, thereby enhancing comfortability when wearing the earplug 10′.

On the contrary, when the earplug 10′ is gradually separated from the person's ear canal 60, the pressure produced by the ear wall on the compression portion 22′ also gradually decreases along with a correspondingly gradual decrease in the pushing force of the filler material 40′ on the air, at which time the pressure within the air chamber 27′ is less than the atmospheric pressure outside the person's ear canal 60, which causes air to gradually enter the air chamber 27′ through the air passage 34′ and compress the filler material 40′ until the earplug 10′ is completely separated from the person's ear canal 60, whereupon the filler material 40′ continues to be compressed by the air, causing the compression portion 22′ to return to its original state from a compressed state, thereby further providing the earplug 10′ with the effect of reusability and substantially extending the serviceable life thereof.

It is worth mentioning that it is of prime importance to hold a holding section 35′ of the supporting stem 30′ when inserting/extracting the earplug 10′ into/out of the person's ear canal 60 to ensure added convenience when carrying out insertion or extraction of the earplug 10′.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. An earplug with enhanced wearing comfortability, comprising:
a main guide body, provided with a guide portion, a compression portion that extends from the guide portion, and a stop portion that extends from the compression portion, wherein a connecting cavity is provided inside the guide portion, a filling cavity is provided inside the compression portion, and an opening portion is provided inside the stop portion; moreover, the filling cavity interconnects the connecting cavity and the opening portion;
a supporting stem, which is provided with a connecting section that enables embedding into the connecting cavity, an extended section that extends from the connecting section and is positioned inside the filling cavity, and a securing section that extends from the extended section and embeds within the opening portion; moreover, the securing section is provided with an air passage, which enables air to pass therethrough from an exterior of the main guide body and circulate to an interior of the filling cavity; and
a filler material provided with plasticity, which fills the interior of the filling cavity and causes an air chamber to form therein and contain air, such that, air enters the air chamber through the air passage and cause the compression portion to form its original state;
whereby when the compression portion is pressed and constricted, the compression portion compresses the air within the air chamber through the filler material, thereby expelling the air through the air passage and causing the compression portion to form a compressed state; and after releasing the pressure on the compression portion, air enters the air chamber through the air passage and squeezes the filler material, thereby causing the compression portion to return to an original state from a compressed state.

2. The earplug with enhanced wearing comfortability according to claim 1, wherein the compression portion is further assembled from a first compression area connected to the guide portion and a second compression area that extends from the first compression area and connects with the stop portion.

3. The earplug with enhanced wearing comfortability according to claim 2, wherein the filling cavity is further assembled from a first filling cavity that is positioned inside the first compression area and a second filling cavity that is positioned inside the second compression area and connects with the air passage; moreover, the first filling cavity and the second filling cavity are interconnected.

4. The earplug with enhanced wearing comfortability according to claim 3, wherein the filler material fills the first filling cavity and a portion of the second filling cavity, thereby causing an air chamber to form inside the second filling cavity.

5. The earplug with enhanced wearing comfortability according to claim 1, wherein a mutually embedding structure is formed between the opening portion and the securing section.

6. The earplug with enhanced wearing comfortability according to claim 5, wherein the embedding structure comprises a first embedding portion positioned at the opening portion and a second embedding portion positioned on the securing section and embeds into the first embedding portion.

7. The earplug with enhanced wearing comfortability according to claim 1, wherein both the guide portion and the stop portion assume an umbrella form.

8. The earplug with enhanced wearing comfortability according to claim 1, wherein the filler material is formed from silicone material.

9. The earplug with enhanced wearing comfortability according to claim 1, wherein the supporting stem further comprises a holding section extending from the securing section toward an outer side of the opening portion, and the holding section extends to the exterior of the main guide body.
